(19) United States
(12) Patent Application Publication
(10) Pub. No.: US 2004/0142474 A1
(43) Pub. Date: Jul. 22, 2004

(54) Novel cationic lipopolymer as a biocompatible gene delivery agent

(75) Inventors: Ram I. Mahato, Cordova, TN (US); Sang-Oh Han, Huntsville, AL (US); Darin Y. Furgeson, Carrboro, NC (US); Khursheed Anwer, Madison, AL (US)

Correspondence Address: Weili Cheng, THORPE NORTH & WESTERN, LLP, P.O. Box 1219, Sandy, UT, 84091-1219, US

(73) Assignee: Expression Genetics, Inc.

(21) Appl. No.: 10/717,109

(22) Filed: Nov. 19, 2003

Related U.S. Application Data
(63) Continuation-in-part of application No. 10/083,861, filed on Feb. 25, 2002.
Application No. 10/083,861 is a continuation-in-part of application No. 09/662,511, filed on Sep. 14, 2000, now Pat. No. 6,696,038.

(51) Int. Cl. C08G 63/48; C08G 63/91; C12N 15/88

(52) U.S. Cl. 435/458; 525/54.1; 525/54.2

(57) ABSTRACT

A biodegradable cationic lipopolymer comprising a polyethylenimine (PEI), a lipid, and a biocompatible hydrophilic polymer, wherein 1) the lipid and the biocompatible hydrophilic polymer are directly linked to the PEI backbone or 2) the lipid is linked to the PEI backbone through the biocompatible hydrophilic polymer. The cationic lipopolymers of the present invention can be used for delivery of a nucleic acid or any anionic bioactive agent to various organs and tissues after local or systemic administration.

This application is a continuation-in-part of pending U.S. patent application Ser. No. 10/083,861, filed Feb. 25, 2002, which in turn is a continuation-in-part of pending U.S. patent application Ser. No. 09/662,511, filed Sep. 4, 2000

### BACKGROUND OF THE INVENTION

1. Field of the Invention

This invention relates generally to cationic lipopolymers and methods of preparing thereof. It relates particularly to a biodegradable cationic lipopolymer comprising a polyethylenimine (PEI), a lipid, a biocompatible hydrophilic polymer, wherein: 1) the lipid and the biocompatible hydrophilic polymer are directly linked to the PEI backbone or 2) the lipid is linked to the PEI backbone through the biocompatible hydrophilic polymer. The cationic lipopolymers of the present invention are useful for the delivery of a nucleic acid or an anionic agent into cells.

2. Related Art

Gene therapy is generally considered as a promising approach not only for the treatment of diseases with genetic defects, but also in the development of strategies for treatment and prevention of chronic diseases such as cancer, cardiovascular disease and rheumatoid arthritis. However, nucleic acids as well as other polyanionic substances are rapidly degraded by certain enzymes and exhibit poor cellular uptake when delivered in aqueous solutions. Since early efforts to identify methods for delivery of nucleic acids into tissues or culture cells in the mid 1950's, steady progress has been made towards improving delivery of functional DNA, RNA, and antisense oligonucleotides both in vitro and in vivo.

The gene carriers used so far include viral systems (retroviruses, adenoviruses, adeno-associated viruses, or herpes simplex viruses) or nonviral systems (liposomes, polymers, peptides, calcium phosphate precipitation and electroporation). Viral vectors have been shown to have high transfection efficiency when compared to nonviral vectors, but their use in vivo is severely limited due to several drawbacks, such as dependence on cell division, risk of random DNA insertion into the host genome, low capacity for carrying large sized therapeutic genes, risk of replication, and possible host immune reaction.

Compared to viral vectors, nonviral vectors are easy to make and are less likely to produce immune reactions. In addition, there is no replication reaction required. There has been increasing attention focused on the development of safe and efficient nonviral gene transfer vectors, which are either cationic lipids or polycationic polymers. Polycationic polymers such as poly-L-lysine, poly-L-ornithine and polyethylenimine (PEI) that interact with DNA to form polyionic complexes have been introduced for use in gene delivery. Various cationic lipids have also been shown to form lipoplexes with DNA and induce transfection of various eukaryotic cells. Many different cationic lipids are commercially available and several have already been used in the clinical setting. Although the mechanism of lipid transfection is not yet clear, it probably involves binding of the DNA/lipid complex with the cell surface via excess positive charges on the complex and release of DNA into cytoplasm from the endosome formed. Cell surface bound complexes are probably internalized and the DNA released into the cytoplasm of the cell from an endocytic compartment.

However, it is not feasible to directly extend in vitro transfection technology for in vivo application. Relative to in vivo use, the biggest drawback of the diether lipids, such as N-[1-(2,3-dioleyloxy)propyl]-N,N,N-trimethyl ammonium chloride (DOTMA) or Lipofectin, is that they are not natural metabolites of the body, and are thus not biodegradable. They are also toxic to cells. In addition, it has been reported that cationic lipid transfection is inhibited by factors present in serum and thus they are an ineffective means for the introduction of genetic material into cells in vivo. In addition, these cationic lipids have been proven less efficient in in vivo gene transfer.

An ideal transfection reagent should exhibit a high level of transfection activity without needing any mechanical or physical manipulation of cells or tissues. The reagent should be nontoxic, or minimally toxic, at the effective dose. In order to avoid any long-term adverse side effects on the treated cells, it should also be biodegradable. When gene carriers are used for delivery of nucleic acids in vivo, it is essential that the gene carriers themselves are nontoxic and that they degrade into nontoxic products. To minimize the toxicity of the intact gene carrier and its degradation products, the design of gene carriers needs to be based on naturally occurring metabolites.

U.S. Pat. No. 5,283,185, Epand et al. (hereafter the '185 patent), discloses a method for facilitating the transfer of nucleic acids into cells comprising preparing a mixed lipid dispersion of a cationic lipid, 3′[N-(N′,N″-dimethylaminoethane)carbamoyl]cholesterol (DC-cholesterol) with a co-lipid in a suitable carrier solvent. The method disclosed in the '185 patent involves using a halogenated solvent in preparing a liposome suspension. In pharmaceutical applications, residues of halogenated solvents cannot be practically removed from a preparation after having been introduced. U.S. Pat. No. 5,753,262, (hereafter the '262 patent) discloses using the acid salt of the lipid 3′[N-(N′,N″-dimethylaminoethane)-carbamoyl]cholesterol (DC-cholesterol) and a helper lipid, such as dioleoyl phosphatidylethanolamine (DOPE) or dioleoylphosphatidylcholine (DOPC), to produce effective transfection in vitro.

Because of their sub-micron size, nanoparticles are hypothesized to enhance interfacial cellular uptake, thus achieving in a true sense a “local pharmacological drug effect.” It is also hypothesized that there would be enhanced cellular uptake of drugs contained in nanoparticles (due to endocytosis) compared to the corresponding free drugs. Nanoparticles have been investigated as drug carrier systems for tumor localization of therapeutic agents in cancer therapy, for intracellular targeting (antiviral or antibacterial agents), for targeting to the reticuloendothelial system (parasitic infections), as immunological adjuvants (by oral and subcutaneous routes), for ocular delivery with sustained drug action, and for prolonged systemic drug therapy.

In view of the foregoing, it will be appreciated that providing a gene carrier that is biodegradable, capable of forming nanoparticles, liposomes, or micelles, and that is able to escape the immune system and so provide for safe and efficient gene delivery, is desired. The novel cationic lipopolymer of the present invention comprises a polyethylenimine (PEI), a lipid, and a biocompatible hydrophilic polymer, wherein the lipid is covalently bound to the PEI backbone directly or through a hydrophobic polymer spacer, which in turn is covalently bound to a primary or secondary amine group of the PEI.

The lipopolymer of the present invention is useful for preparing cationic micelles or cationic liposomes for delivery of nucleic acids or other anionic bioactive molecules, or both, and is readily susceptible to metabolic degradation after incorporation into the cell.

### SUMMARY OF THE INVENTION

It has been recognized that it would be advantageous to develop a biodegradable cationic lipopolymer, having reduced in vivo and in vitro cellular toxicity, for delivery of nucleic acids. The lipopolymers of the present invention can effectively carry out both stable and transient transfection into cells of polynucleotide such as DNA and RNA.

In accordance with more detailed aspects of the present invention, the cationic lipopolymers of the present invention comprise a polyethylenimine (PEI), a lipid, and a biocompatible hydrophilic polymer, wherein: 1) the lipid and the biocompatible hydrophilic polymer are directly linked to the PEI backbone or 2) the lipid is linked to the PEI backbone through the biocompatible hydrophilic polymer. The PEI is either branched or linear in configuration, with an average molecular weight within the range of 100 to 500,000 Daltons. The covalent bond between the PEI, the hydrophilic polymer and the lipid is preferably a member selected from the group consisting of an ester, amide, urethane and di-thiol bond. The hydrophilic polymer is preferably a polyethylene glycol (PEG) having a molecular weight of between 50 to 20,000 Daltons. The molar ratio of the PEI to the conjugated lipid is preferably within a range of 1:0.1 to 1:500. The cationic lipopolymers of the present invention may further comprise a targeting moiety.

The cationic lipopolymers of the present invention can be prepared as liposomes or water soluble micelles depending upon their coformulation with neutral lipids, such as DOPE or cholesterol. For example, in the presence of neutral lipids the lipopolymers will form water insoluble liposomes, and in the absence of neutral lipids the lipopolymers will form water soluble micelles.

The cationic lipopolymers of the present invention can spontaneously form discrete nanometer-sized particles with a nucleic acid, which can promote more efficient gene transfection into mammalian cell lines than can be achieved conventionally with Lipofectin and polyethylenimine. The lipopolymers of the present invention are readily susceptible to metabolic degradation after incorporation into animal cells. The biocompatible and biodegradable cationic lipopolymers of this invention provide improved gene carriers for use as a general reagent for transfection of mammalian cells, and for the in vivo applications of gene therapy.

The present invention further provides transfection formulations, comprising a novel cationic lipopolymer, complexed with a selected nucleic acid in the proper charge ratio (positive charge of the lipopolymer/negative charge of the nucleic acid) such that it is optimally effective for both in vivo and in vitro transfection. The N/P (nitrogen atoms to polymer/phosphate atoms on the DNA) ratio of the cationic lipopolymer and the nucleic acid is preferably within the range of 500/1 to 0.1/1. Particularly, for systemic delivery, the N/P ratio is preferably 1/1 to 100/1; for local delivery, the N/P ratio is preferably 0.5/1 to 50/1.

This invention also provides for a method of transfecting, both in vivo and in vitro, a nucleic acid into a mammalian cell. The method comprises contacting the cell with cationic lipopolymers or liposome:nucleic acid complexes as described above. In one embodiment the method uses the cationic lipopolymer/DNA complexes for local delivery into a warm blooded animal. In a particularly preferred embodiment, the method comprises local administration of the cationic lipopolymer/DNA complexes into solid tumors in a warm blooded animal. In another embodiment, the method uses systemic administration of the cationic lipopolymer or liposome:nucleic acid complex into a warm-blooded animal. In a preferred embodiment, the method of transfecting uses intravenous administration of the cationic lipopolymer or liposome:nucleic acid complex into a warm-blooded animal. In a particularly preferred embodiment, the method comprises intravenous injection of water soluble lipopolymer/pDNA, lipopolymer:DOPE liposome/pDNA or lipopolymer:cholesterol liposome/pDNA complexes into a warm blooded animal.

### BRIEF DESCRIPTION OF THE DRAWINGS

FIG. 1 illustrates a synthetic scheme to prepare a lipopolymer of PEG-PEI-Cholesterol (PPC) where the lipid (cholesterol) and hydrophilic polymer (PEG) are directly linked to the PEI backbone through a covalent linkage.

FIG. 2. illustrates determination of the chemical structure by ¹H NMR of the PEG-PEI-Cholesterol lipopolymer consisting of branched PEI 1800, Cholesteryl chloroformate and PEG 550 (FIG. 2A) or PEG 330 by (FIG. 2B).

FIG. 3 illustrates determination by ¹HNMR of the chemical structure of the PEG-PEI-cholesterol lipopolymer consisting of linear PEI 25000, PEG 1000 and Cholesterol chloroformate.

FIG. 4 illustrates gel retardation assays of PEG-PEI-Cholesterol (1:1:1 ratio)/pDNA complexes according at various N/P ratios A: naked pDNA, B:WSLP2 (N/P=20/1), C:WSLP0331 (N/P=20/1), D:WSLP0405 (N/P=20/1), E: PPC(N/P=10/1), F: PPC(N/P=15/1), G:PPC(N/P=17/1), H: PPC (20/1), I: PPC(N/P=30/1), J: PPC (40/1), and K: PPC (consisting 0.2 moles PEG, 1 mole PEI, and 1 Mole cholesterol) (N/P=20/1).

FIG. 5 illustrates the physicochemical properties (surface charge by zeta potential (left bar) and particle size (right bar)) of PPC/pDNA complexes at various N/P ratios.

FIG. 6 illustrates luciferase gene transfer into cultured human embryonic kidney transformed cells (293 T cells) after transfection with PPC/pDNA complexes at different PEG to PEI ratios (1-2.5).

FIG. 7 illustrates luciferase gene transfer into subcutaneous 4T1 tumors after transfection with PPC/pCMV-Luc complexes at various PEG to PEI ratios.

FIG. 8 illustrates mIL-12 gene transfer into subcutaneous 4T1 tumors after intratumoral injection of PPC/pDNA complexes in BALB/c mice.

FIG. 9 illustrates luciferase gene transfer into mouse lungs by PPC liposome/pDNA complexes after intravenous administration

FIG. 10 illustrates inhibition of mouse lung tumors by PPC liposome/mL-12 pDNA complexes after intravenous administration.

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

Before the present composition and method for delivery of a bioactive agent are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a polymer containing “a bond” includes reference to two or more of such bonds. In describing and claiming the present invention, the following terminology will be used in accordance with the definitions set out below.

“Transfecting” or “transfection” shall mean transport of nucleic acids from the environment external to a cell to the internal cellular environment, with particular reference to the cytoplasm and/or cell nucleus. Without being bound by any particular theory, it is understood that nucleic acids may be delivered to cells either in the form of or after being encapsulated within or adhering to one or more cationic lipid/nucleic acid complexes or be entrained therewith. Particular transfecting instances deliver a nucleic acid to a cell nucleus. Nucleic acids include DNA and RNA as well as synthetic congeners thereof. Such nucleic acids include missense, antisense, nonsense, as well as protein producing nucleotides, on and off, and rate regulatory nucleotides that control protein, peptide, and nucleic acid production. In particular, but not limiting, they can be genomic DNA, cDNA, mRNA, tRNA, rRNA, hybrid sequences or synthetic or semi-synthetic sequences and of natural or artificial origin. In addition, the nucleic acid can be variable in size, ranging from oligonucleotides to chromosomes. These nucleic acids may be of human, animal, vegetable, bacterial, viral, and the like, origin. They may be obtained by any technique known to a person skilled in the art.

As used herein, the term “bioactive agent” or “drug” or any other similar term means any chemical or biological material or compound, suitable for administration by the methods previously known in the art and/or by the methods taught in the present invention, which will induce a desired biological or pharmacological effect. These effects may include but are not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect may be local, such as providing for a local anesthetic effect, or it may be systemic.

As used herein, “effective amount” means an amount of a nucleic acid and/or an anionic agent that is sufficient to form a biodegradable complex with the cationic lipopolymers of the present invention and allow for delivery of the nucleic acid or anionic agent into cells.

As used herein, a “liposome” means a microscopic vesicle composed of uni-or multi-layers surrounding aqueous compartments.

As used herein, “administering,” and similar terms mean delivering the composition to the individual being treated such that the composition is capable of being circulated systemically where the composition binds to a target cell and is taken up by endocytosis. Thus, the composition is preferably administered systemically to the individual, typically by subcutaneous, intramuscular, intravenous, or intraperitoneal injection. Injectables for such use can be prepared in conventional forms, either as a liquid solution, suspension, or in a solid form that is suitable for preparation as a solution or suspension in a liquid prior to injection, or as an emulsion. Suitable excipients include, for example, water, saline, dextrose, glycerol, ethanol, and the like; and if desired, minor amounts of auxiliary substances such as wetting or emulsifying agents, buffers, and the like can be added.

Fundamental to the success of gene therapy is the development of gene delivery vehicles that are safe and efficacious for systemic administration. Many of the cationic lipids used in the early clinical trials, such as N[1-(2,3-dioleyloxy)propyl]-N,N, N-trimethylammonium chloride (DOTMA) and 3-β(N,N″-dimethylaminoethane carbamoyl cholesterol) (DC-Chol), although exhibiting efficient gene transfer in vitro, have been proven to be less efficient in gene transfer in animals. See Felgner P L et al. Lipofection: A highly efficient, lipid-mediated DNA transfection procedure. Proc Natl Acad Sci USA 84: 7413-7417 (1987); and Gao, X. and Huang L. (1991) A novel cationic liposome reagent for efficient transfection of mammalian cells. Biochem. Biophys. Res. Commun. 179: 280-285.

The general structure of a cationic lipid has three parts: (i) a hydrophobic lipid anchor, which helps in forming liposomes (or micellar structures) and interacts with cell membranes; (ii) a linker group; and (iii) a positively charged head-group, which interacts with the plasmid, leading to its condensation. Many compounds bearing either a single tertiary or quaternary ammonium head-group or which contain protonatable polyamines linked to dialkyl lipids or cholesterol anchors have been used for transfection into various cell types. The orientation of the polyamine head-group in relation to the lipid anchor has been shown to greatly influence the transfection efficiency. Conjugation of spermine or spermidine head-groups to the cholesterol lipid via a carbamate linkage through a secondary amine, to generate T-shaped cationic lipids, has been shown to be very effective in gene transfer in lung tissue. In contrast, a linear polyamine lipid formed by conjugating spermine or spermidine to cholesterol or a dialkyl lipid was much less effective in gene transfer.

A cationic lipid which contains three protonatable amines in its head-group has been shown to be more active than DC-Cholesterol, which contains only one protonatable amine. In addition to the number of protonatable amines, the choice of the linker group bridging the hydrophobic lipid anchor with the cationic head-group has also been shown to influence gene transfer activity. Substitution of a carbamate linker with, urea, amide, or amine, results in an appreciable loss of transfection activity. PEI has been shown to be highly effective in gene transfer, which is dependent on its molecular weight and charge ratio. However, high molecular weight PEI is very toxic to cells and tissues.

The cationic lipopolymer of the present invention comprises a polyethylenimine (PEI), a lipid, and a biocompatible hydrophilic polymer, wherein the lipid and the hydrophilic polymer are covalently bound to PEI backbone. Optionally, the lipid can be covalently bound to the PEI via a hydrophilic polymer spacer. Preferably, the hydrophilic polymer is polyethylene glycol (PEG) having a molecular weight of between 50 to 20,000 Daltons. Preferably, the lipid is cholesterol, cholesterol derivatives, C₁₂ to C₁₈ fatty acids, or C₁₂ to C₁₈ fatty acid derivatives. The lipopolymer of the present invention is characterized in that one or more lipids and hydrophilic polymers are conjugated to the PEI backbone.

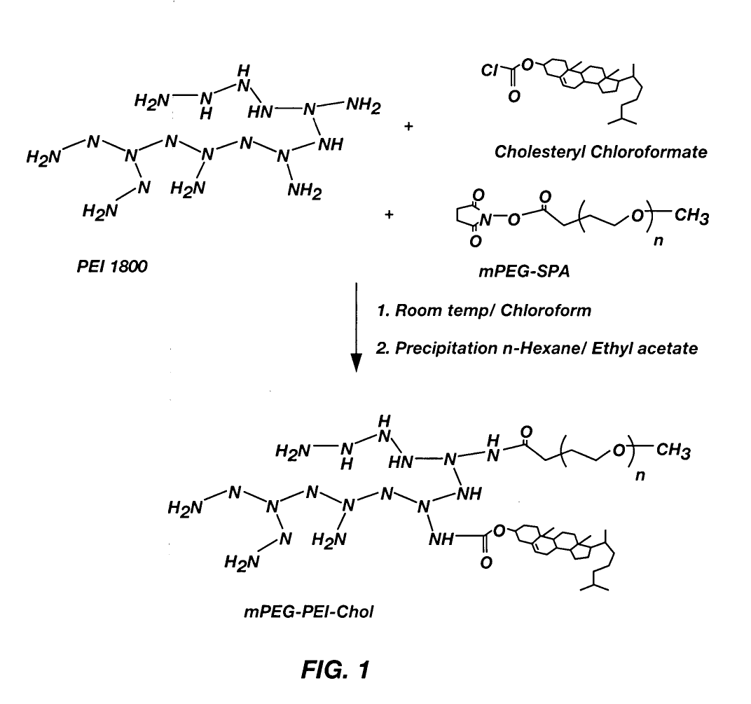

FIG. 1 illustrates the synthetic scheme of the lipopolymer of the present invention. The detailed synthesis procedure is as follows: One gram of branched polyethyleneimine (PEI) 1800 Da (0.56 mM) was dissolved in 5 ml chloroform and placed in a 100 ml round bottom flask and stirred for 20 minutes at room temperature. Three hundred eighty milligrams of cholesteryl chloroformate (0.85 mM) and 500 mg poly(ethylene glycol)(PEG) (mw 550 Da)(0.91 mM) was dissolved in 5 ml chloroform and transferred to an addition funnel which was located on the top of the round bottom flask of the PEI solution. The mixture of Cholesteryl chloroformate and PEG in chloroform was slowly added to the PEI solution over 5-10 minutes at room temperature and then stirred for additional 4 hrs at room temperature. After removing the solvent from the reaction mixture by rotary evaporator, the remaining sticky material was dissolved in 20 ml ethyl acetate with stirring. The product was precipitated from the solvent by slowly adding 20 ml of n-Hexane, and then the liquid was decanted from the product. The product was washed two times with 20 ml of a mixture of ethyl acetate/n-Hexane (1/1; v/v). After decanting the liquid, the material was dried by purging nitrogen gas for 10-15 minutes. The material was dissolved in 10 ml 0.05N HCl to obtain the salt form of the amine groups since the free base from is easily oxidized when coming in contact with air. The aqueous solution was filtered through a 0.2 μm filter paper and then lyophilized to obtain the final product.

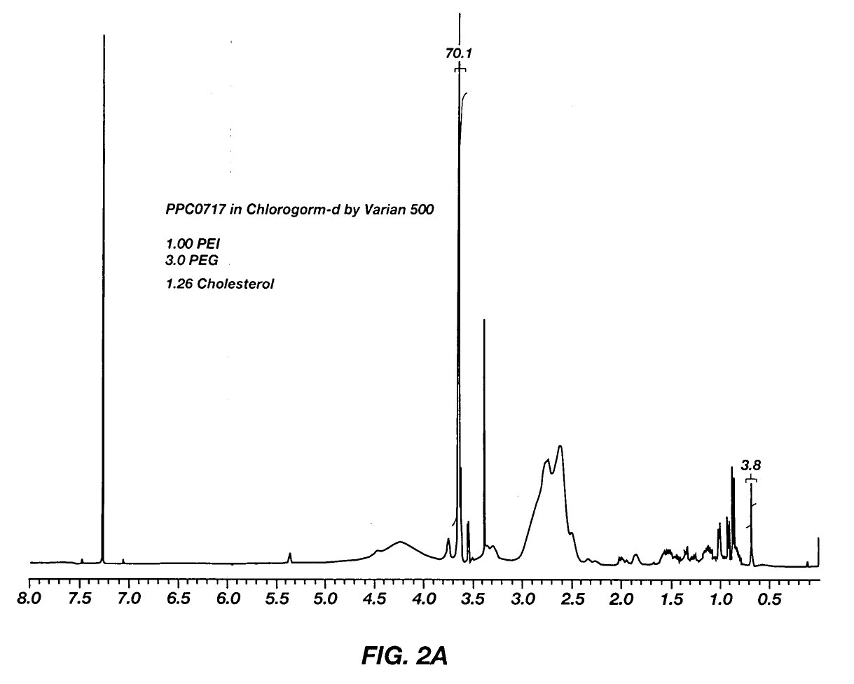

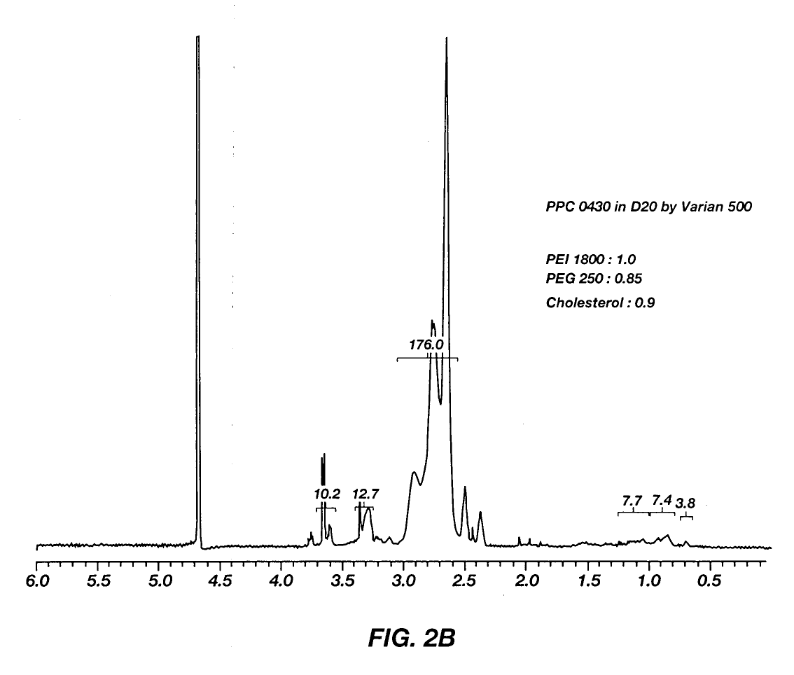

The identity of the final product (presence Cholesterol, PEG, and PEI) was confirmed by. ¹H-NMR (Varian Inc., 500 MHz, Palo, Alto, Calif.). The NMR results are as follows: ¹H NMR (500 MHz, chloroform-d1) δ ˜0.65 ppm (3H of CH₃ from cholesterol (a)); δ ˜0.85 ppm (6H of (CH₃)₂ from cholesterol); δ ˜0.95 ppm (3H of CH₃ from cholesterol); δ˜ 1.110 ppm (3H of CH₃ from cholesterol); δ0.70˜2.50 ppm (4H from CH₂—CH₂ and CHCH₂ from cholesterol); δ˜ 5.30 ppm (1H from ═CH— from cholesterol); δ2.50˜3.60 ppm (176H from N—CH₂—CH₂—N from PEI (b)); and δ ˜3.7 ppm (23H from OCH₂CH₂—O from PEG(c)). The representative peaks of each material (marked (a), (b), and (c)) was calculated by dividing the number of hydrogens, and then calculating the conjugation ratios (FIG. 2A). The molar ratio of this example showed that 3.0 moles PEG and 1.28 moles cholesterol were conjugated to one mole of PEI molecules.

A second approach to PPC synthesis involves using PEG 250 Da, PEI 1800 and cholesteryl chloroformate to obtain a PPC with 0.85 moles of PEG and 0.9 moles of cholesteryl chloroformate to 1.0 mole of PEI molecules, as illustrated in FIG. 2B. This demonstrates that a broad molecular weight range of PEG can be used for PPC synthesis.

In another conjugation approach, linear polyethylenimine (LPEI) was utilized for PPC synthesis. Although branched PEI has three different kinds of amines (approximately 25% primary amines, 50% secondary amines, and 25% tertiary amines), linear PEI consists of only secondary amines. Therefore, a cholesterol derivative and PEG were conjugated to the secondary amines of linear PEI. The detailed synthesis and analysis methods are as follows. Five hundred milligrams of LPEI (mw 25000 Da) (0.02 mM) was dissolved in 30 ml chloroform at 65° C. for 30 minutes. A mixture of 40 mg cholesteryl chloroformate (0.09 mM) and 200 mg PEG (mw 1000 Da) (0.2 mM) in 5 ml chloroform was slowly added to the PEI solution over 3-10 minutes. The solution was stirred for an additional 4 hrs at 65° C. The solvent was removed under vacuum by a rotary evaporator, and the remaining materials were washed with 15 ml of ethyl ether. After drying with pure nitrogen, the material was dissolved in a mixture of 10 ml of 2.0 N HCl and 2 ml of trifluoroacetic acid. The solution was dialyzed against deionized water using a MWCO 15000 dialysis tube for 48 hrs with changing fresh water every 12 hrs. The solution was lyophilized to remove the water.

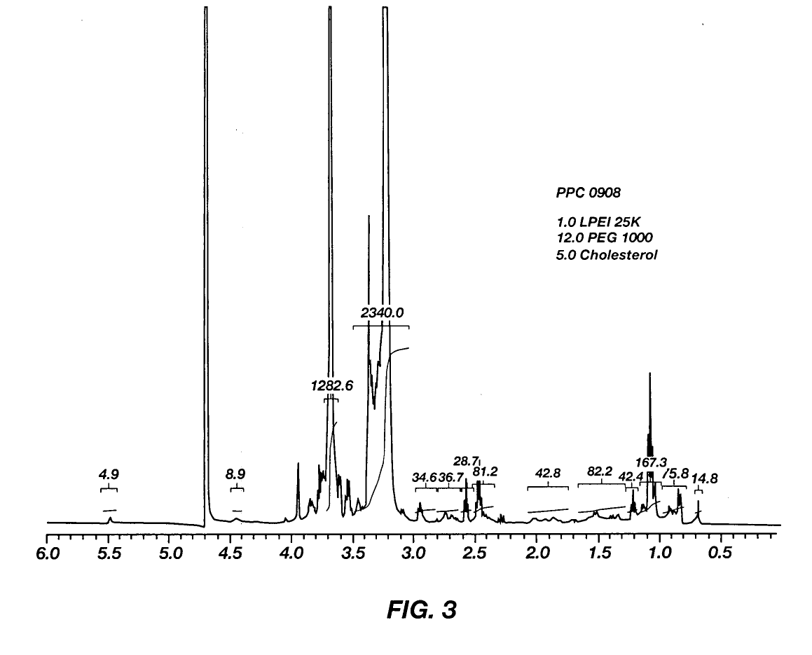

For confirmation of the product composition, the final product was analyzed by ¹H-NMR (Varian Inc., 500 MHz, Palo, Alto, Calif.). A sample was dissolved in deuterium oxide for NMR measurement. The NMR peaks were analyzed by carrying out characterization of the presence of three components, Cholesterol, PEG, and PEI. The NMR results are as follows: ¹H NMR (500 MHz, chloroform-d 1) δ ˜0.65 ppm (3H of CH₃ from cholesterol); (2340H from N—CH₂—CH₂—N from PEI); and δ ˜3.7 ppm (91H from OCH₂CH₂—O from PEG). The representative peaks of each material were calculated by divided the number of hydrogens, and then considered the conjugation ratios. The molar ratio of this example showed that 12.0 moles PEG and 5.0 moles cholesterol were conjugated to one mole of PEI molecules (FIG. 3).

One example of a novel lipopolymer is poly[N-poly(ethylene glycol)-ethyleneimine]-co-poly(ethyleneimine)-co-poly(N-cholesterol) (hereafter as “PPC”). The free amines of the PEI contained in PPC provide sufficient positive charges for adequate DNA condensation. The linkage between the polar head group and hydrophobic lipid is biodegradable and yet strong enough to survive in a biological environment. The ester linkage between the cholesterol lipid and polyethylenimine provides for the biodegradability of the lipopolymer and the relatively low molecular weight PEI significantly decreases the toxicity of the lipopolymer. Although cholesterol derived lipid is preferred in the present invention, other lipophilic moieties may also be used, such as C₁₂ to C₁₈ saturated or unsaturated fatty acids.

The biodegradable cationic lipopolymer of the present invention has amine group(s) which is electrostatically attracted to polyanionic compounds such as nucleic acids. The cationic lipopolymer of the present invention condenses DNA, for example, into compact structures. Upon administration, such complexes of these cationic lipopolymers and nucleic acids are internalized into cells through receptor mediated endocytosis. In addition, the lipophilic group of the lipopolymer allows the insertion of the cationic amphiphile into the membrane of the cell and serves as an anchor for the cationic amine group to attach to the surface of the cell. The lipopolymers of the present invention have both highly charged positive group(s) and hydrophilic group(s), which greatly enhance cellular and tissue uptake during the delivery of genes and other bioactive agents.

Instability of condensed nucleic acids under physiological conditions is one of the major hurdles for their clinical use. The other major limitation to the in vivo use of condensed nucleic acids is their tendency to interact with serum proteins, resulting in destabilization and rapid clearance by reticuloendothelial cells following intravenous administration. The compatibility and solubility of cationic lipopolymers can be improved by conjugation with hydrophilic biocompatible polymers like poly(ethylene glycol) (PEG). PEG is an FDA-approved polymer known to inhibit the immunogenicity of molecules to which it is attached. PEGylation covers the condensed DNA particles with a “shell” of the PEG, stabilizes the nucleic acids against aggregation, decreases recognition of the cationic lipopolymer by the immune system, and slows their breakdown by nucleases after in vivo administration.

The amine groups on the PEI can also be conjugated with the targeting moiety via spacer molecules. The targeting moiety conjugated to the lipopolymer directs the lipopolymer-nucleic acid/drug complex to bind to specific target cells and penetrate into such cells (tumor cells, liver cells, heamatopoietic cells, and the like). The targeting moiety can also be an intracellular targeting element, enabling the transfer of the nucleic acid/drug to be guided towards certain favored cellular compartments (mitochondria, nucleus, and the like). In a preferred embodiment, the targeting moiety can be a sugar moiety coupled to the amino groups. Such sugar moieties are preferably mono- or oligosaccharides, such as galactose, glucose, fucose, fructose, lactose, sucrose, mannose, cellobiose, triose, dextrose, trehalose, maltose, galactosamine, glucosamine, galacturonic acid, glucuronic acid, and gluconic acid. Preferably, the targeting moiety is a member selected from the group consisting of transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents such as polymixin B and hemagglutinin HA2, lysosomotrophic agents, and nucleus localization signals (NLS).

Conjugation of the acid derivative of a sugar with the cationic lipopolymer is most preferred. In a preferred embodiment of the present invention, lactobionic acid (4-O-αZD-galactopyranosyl-D-gluconic acid) is coupled to the lipopolymer. The galactosyl unit of lactose provides a convenient targeting molecule for hepatocytes because of the high affinity and avidity of the galactose receptor on these cells.

An advantage of the present invention is that it provides a gene carrier wherein the particle size and charge density are easily controlled. Control of particle size is crucial for optimization of a gene delivery system because the particle size often governs the transfection efficiency, cytotoxicity, and tissue targeting in vivo. In general, in order to enable its effective penetration into tissue, the size of a gene delivery particle should not exceed the size of clathrin-coated pits on the cell surface. In the present invention, the physico-chemical properties of the lipopolymer/DNA complexes, such as particle size, can be varied by formulating the lipopolymer with a neutral lipid and/or varying the PEG content.

In a preferred embodiment of the invention, the particle sizes will range from about 40 to 400 nm depending on the cationic lipopolymer composition and the mixing ratio of the components. It is known that particles, nanospheres, and microspheres of different sizes when injected accumulate in different organs of the body depending on the size of the particles. For example, particles of less than 150 nm diameter can pass through the sinusoidal fenestrations of the liver endothelium and become localized in the spleen, bone marrow, and possibly tumor tissue. Intravenous, intra-arterial, or intraperitoneal injection of particles approximately 0.1 to 2.0 μm in diameter leads to rapid clearance of the particles from the blood stream by macrophages of the reticuloendothelial system. The novel cationic lipopolymers of the present invention can be used to manufacture dispersions of controlled particle size, which can be organ-targeted in the manner described herein.

It is believed that the presently claimed composition is effective in delivering, by endocytosis, a selected nucleic acid into hepatocytes mediated by low density lipoprotein (LDL) receptors on the surface of cells. Nucleic acid transfer to other cells can be carried out by matching a cell having a selected receptor thereof with a selected targeting moiety. For example, the carbohydrate-conjugated cationic lipids of the present invention can be prepared from mannose for transfecting macrophages, from N-acetyllactosamine for transfecting T cells, and galactose for transfecting colon carcinoma cells.

One example of the present invention comprises a polyethyleneimine (PEI), a lipid, and a biocompatible hydrophilic polymer, wherein the lipid and the hydrophilic polymer are covalently bound to the PEI backbone directly, or a certain lipid can be covalently attached to the PEI through a hydrophilic polymer spacer. The PEI may be a branched or linear configuration. Preferably, the average molecular weight of the PEI is within a range of 100 to 500,000 Daltons. The PEI is preferably conjugated to the lipid and the hydrophilic polymer by an ester, amide, urethane or di-thiol bond. The biocompatible hydrophilic polymer is preferably a polyethylene glycol (PEG) having a molecular weight of between 50 to 20,000 Daltons. The cationic lipopolymer of the present invention may further comprise a targeting moiety. The molar ratio of the PEI to the conjugated lipid is preferably within a range of 1:0.1 to 1:500. Whereas, the molar ratio of the PEI to the conjugated PEG is preferably within a range of 1:0.1 to 1:50.

The water soluble cationic lipopolymers of the present invention are dispersible in water and form cationic micelles and can therefore be used to manufacture sustained release formulations of drugs without requiring the use of high temperatures or extremes of pH, and, for water-soluble drugs such as polypeptides and oligonucleotide without exposing the drugs to organic solvents during formulation. Such biodegradable cationic lipopolymers are also useful for the manufacture of sustained, continuous release, injectable formulations of drugs. They can act as very efficient dispersing agents and can be administered by injection to give sustained release of lipophilic drugs.

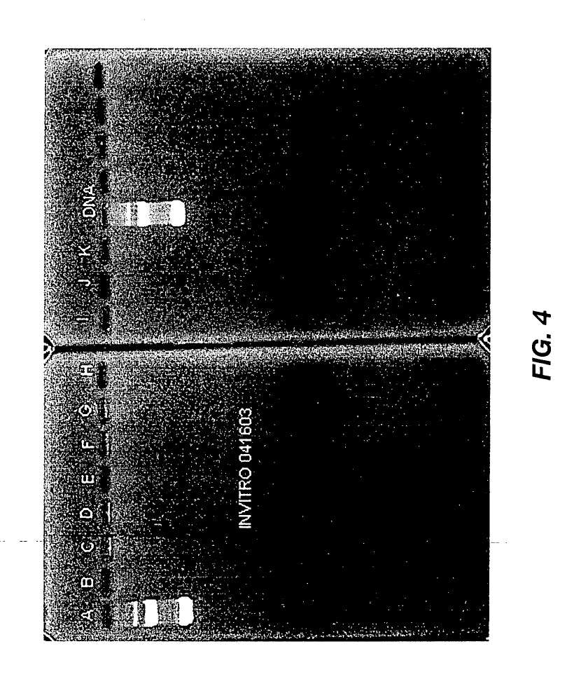

In addition, the lipopolymers of the invention can be used alone or in a mixture with a helper lipid in the form of cationic liposome formulations for gene delivery to particular organs of the human or animal body. The use of neutral helper lipids is especially advantageous when the N/P (amine atoms on polymers/phosphates atoms on DNA) ratio is low. Preferably the helper lipid is a member selected from the groups consisting of cholesterol, dioleoylphosphatidylethanolamine (DOPE), oleoylpalmitoylphosphatidylethanolamin (POPE), diphytanoylphosphatidylethanolamin (diphytanoyl PE), disteroyl-, -palmitoyl-, and -myristoylphosphatidylethanolamine as well as their 1- to 3-fold N-methylated derivatives. Preferably, the molar ratio of the lipopolymer to the helper lipid is within a range of 0.1/1 to 500/1, preferably 0.5/1 to 4/1 and more preferably is within a range of 1/1 to 2/1. To optimize the transfection efficiency of the present compositions, it is preferred to use water as the excipient and diphytanoyl PE as the helper lipid. In addition, the N/P ratio is preferably within the range of 500/1 to 0.1/1, particularly, 100/1 to 1/1 for systemic delivery and 50/1 to 0.5/1 for local delivery. This ratio may be changed by a person skilled in the art in accordance with the polymer used (FIG. 4), the presence of an adjuvant, the nucleic acid, the target cell and the mode of administration used.

Liposomes have been used successfully for transfection of a number of cell types that are normally resistant to transfection by other procedures. Liposomes have been used effectively to introduce genes, drugs, radiotherapeutic agents, enzymes, viruses, transcription factors, and allosteric effectors into a variety of cultured cell lines and animals. In addition, several studies suggest that the use of liposomes is not associated with autoimmune responses, toxicity or gonadal localization after systemic delivery. See, Nabel et al. Gene transfer in vivo with DNA-liposome complexes, Human Gene Ther., 3:649-656, 1992b.

Since cationic liposomes and micelles are known to be good for intracellular delivery of substances other than nucleic acids, the cationic liposomes or micelles formed by the cationic lipopolymers of the present invention can be used for the cellular delivery of substances other than nucleic acids, such as proteins and various pharmaceutical or bioactive agents. The present invention therefore provides methods for treating various disease states, so long as the treatment involves transfer of material into cells. In particular, treating the following disease states is included within the scope of this invention: cancers, infectious diseases, inflammatory diseases and hereditary genetic diseases.

The cationic lipopolymers of the present invention, which show improved cellular binding and uptake of the bioactive agent to be delivered, are directed to overcome the problems associated with known cationic lipids, as set forth above. For example, the biodegradable cationic lipopolymers of the present invention are easily hydrolyzed and the degradation products are small, nontoxic molecules that are subject to renal excretion and are inert during the period required for gene expression. Degradation is by simple hydrolytic and/or enzymatic reaction. Enzymatic degradation may be significant in certain organelles, such as lysosomes. The time needed for degradation can vary from days to months depending on the molecular weight and modifications made to the cationic lipids.

Furthermore, nanoparticles or microsphere complexes can be formed from the cationic lipopolymers of the present invention and nucleic acids or other negatively charged bioactive agents by simple mixing. The lipophilic group (cholesterol derivative) of the cationic lipopolymers of the present invention allows for the insertion of the cationic amphiphile into the membrane of the cell. It serves as an anchor for the cationic amine group to attach to the surface of a cell, which enhances uptake of the cationic carrier/nucleic acid complex by the cell to be transfected. Therefore, the cationic gene carrier of the present invention provides improved transfection efficiency both in vitro and in vivo.

Preferably, a cholesterol moiety is used as a lipophilic portion grafted through a hydrophilic polymer spacer or directly onto the PEI, which serves as a hydrophilic head group in the aqueous environment due to its ionized primary amino groups. As a hydrophilic surface group, the neutral charged PEG can sustain a stable micellar complex that formed a hydrophobic lipid with the hydrophilic head group in the aqueous environment, and provides a shielding effect for the PPC/pDNA complexes against erythrocytes and plasma proteins. In addition, a hydrophilic neutral polymer is essential for enhanced DNA stability in the bloodstream. Whereas, the lipid moiety can be used to enhance the cellular uptake of the DNA complexes by a specific receptor-mediated cell uptake mechanism. Cellular uptake is enhanced by the favorable interaction between the hydrophobic lipid groups and the cellular membrane.

In addition, the neutral charged hydrophilic polymer, such as PEG, provides many advantages for efficient transfection, such as reducing cytotoxicity, improving solubility in aqueous solutions, enhancing stabilization of complexation between the lipopolymer and DNA, and inhibiting interaction between complexes and proteins in blood. In addition, the PEG could prevent interaction between complexes and cell membranes when the complexes are injected into a local site. Therefore, the complexes could distribute well among the cells without easily being captured after administration into local area.

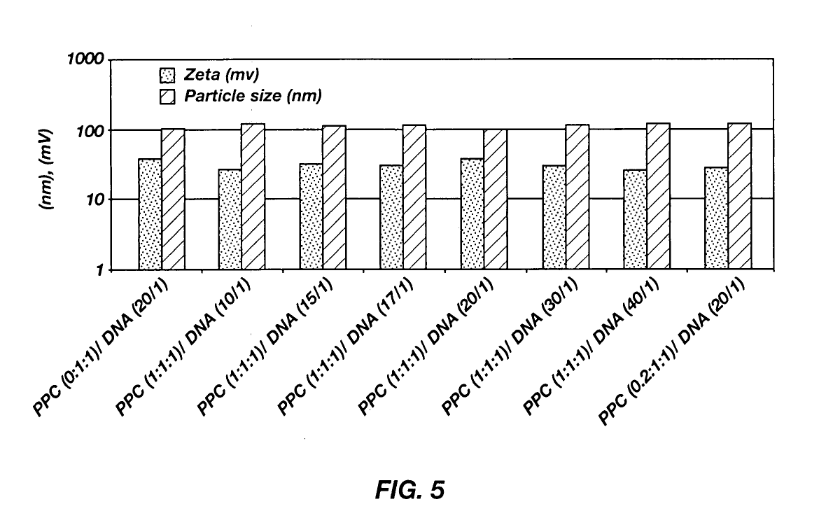

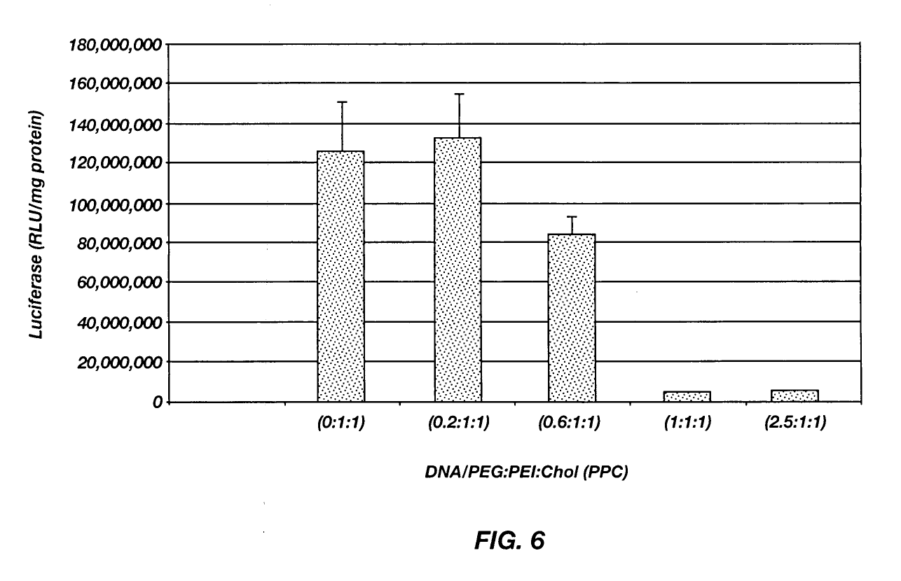

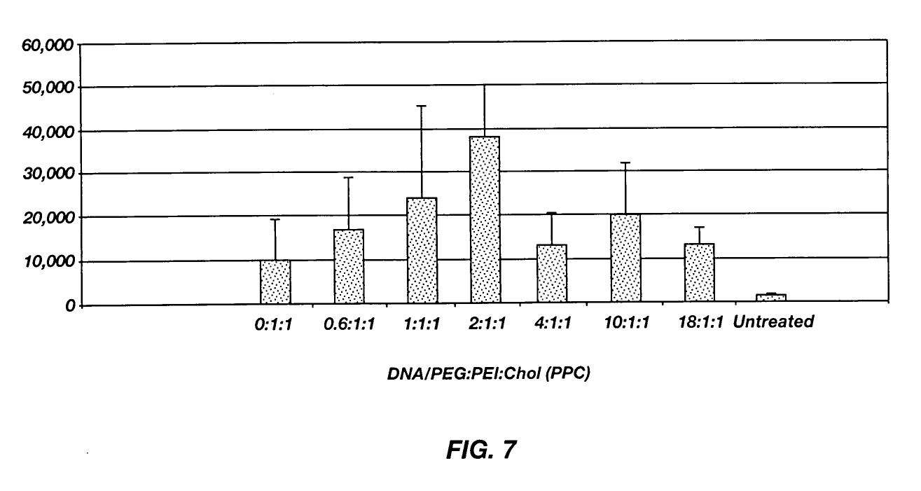

The water soluble lipopolymers of the present invention form micelles and help maintain a delicate balance between the hydrophilic (such as PEI) and hydrophobic (such as cholesterol or fatty acid chains) groups used for complex formation with nucleic acids, which in turn stabilize the DNA/lipopolymer complexes in the bloodstream and improve transfection efficiency. Moreover, water soluble lipopolymers form small size (40˜150 nm) DNA particles (FIG. 5) that are suitable for nucleic acid delivery to hepatocytes or solid tumors. In addition the surface charges of the PPC/pDNA complexes were in a range of 20-40 mV according to N/P ratios showed in FIG. 5. The positively charged particles can easily interact with the negatively charged cell surface. However, despite a net positive charge on the complexes the inclusion of the PEG chain would reduce interaction of the polymer/DNA complexes with the cell membrane thereby yielding lower transfection activity in vitro as the molar ratio of the PEG to the PEI increased. However, the presence of PEG would improve DNA stability in biological milieu producing an overall enhancement in the transfection efficiency of the PPC. As shown in FIG. 6, luciferase activity in cultured 293 T cells was drastically reduced as the PEG/PEI ratios were increased. However, in subcutaneous tumors the luciferase activity increased as PEG/PEI ratio was increased (FIG. 7). The increased in vivo transfection activity of PPC could be due to increased stability and biodistribution of PPC/Luc complexes in biological milieu.

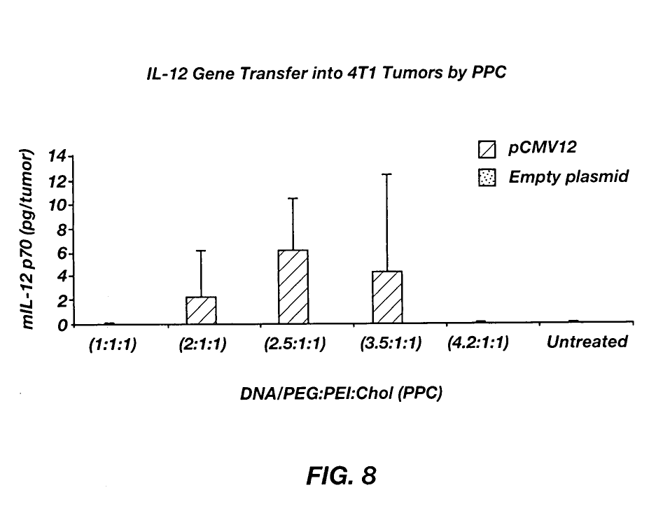

The levels of secreted mIL-12 after transfection of PPC/pmIL-12 complexes were shown to be at the highest level at 3.5 PEG conjugated to each PPC, among the conjugation ratios of 1.0, 2.0, 2.5, 3.5, and 4.2 (FIG. 8). When the result of mIL-12 was compared with luciferase activity on FIG. 7, it could be evaluated that the expression levels of pDNA were not related to the pDNA types but related to the PEG ratio on the PPC as gene carrier.

The effective amount of a composition comprising PPC/pDNA complexes is dependent on the type and concentration of nucleic acids used for a given number and type of cells being transfected. The levels of secreted mIL-12 after intratumoral injection of PPC/pmIL-12 complexes into BALB/c mice bearing 4T 1 subcutaneous tumors was shown to be high when the complexes were composed of PPC with 3.5 moles of PEG conjugated to 1.0 mole PEI and 1.0 mole cholesterol (FIG. 8). Water soluble lipopolymers consisting of PEG, PEI, and cholesterol components are shown to be minimally toxic to cells and tissues after systemic and local administration. PPC and PPC/pDNA complexes were nontoxic to cultured CT-26 colon carcinoma cells, 293 T human embryonic kidney cells and murine Jurkat T-cell lines, even at the higher charge ratios whereas both PEI25000 and LipofectAMINE-based formulations were fairly toxic to these cells.

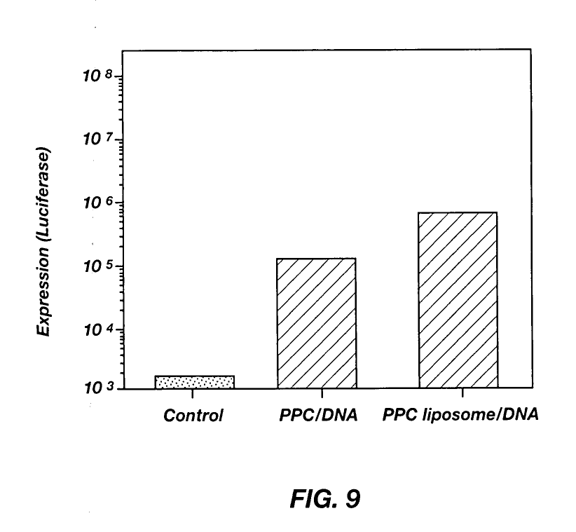

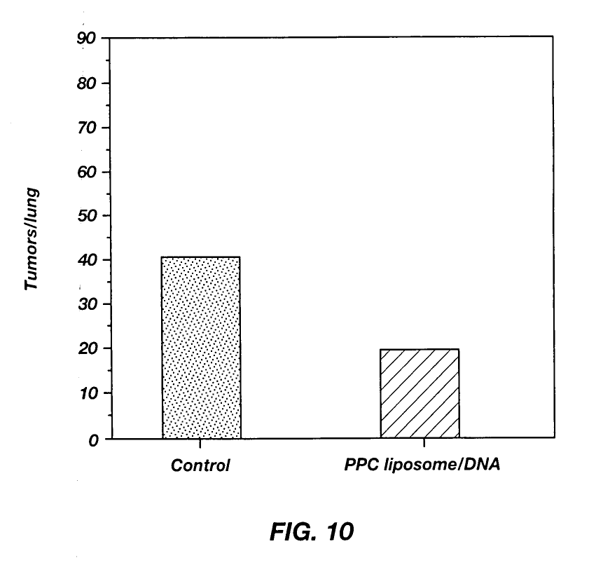

The PPC liposomes form DNA particles of 200-400 nm, which are suitable for nucleic acid delivery to the lung after systemic administration. As shown in FIG. 9, PPC liposomes/luciferase plasmid complexes yielded a 5-10 fold enhancement in lung transfection over a non-liposome formulation of PPC after systemic administration. The transfection efficiency of the PPC liposomes was sufficient to produce therapeutic levels of IL-12 to inhibit the proliferation of tumor nodules in a mouse pulmonary lung metastases model (FIG. 10). The molar ratio of cationic lipopolymer to cholesterol or DOPE affects phase transition of the lipo-particles and the surface chemistry of the lipopolymer:neutral lipid/pDNA complexes. This affects nucleic acid uptake, intracellular decomposition, and trafficking and thus the efficiency of gene expression. The optimal ratio between the lipopolymer and neutral lipid was found to be in the range of 1:1 to 1:2, depending on the target site.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

The following is the general disclosure of the sources of all the chemical compounds and reagents used in the experiments.

Branched polyethylenimine (PEI) of 600, 1200 and 1800 Da, 1,000 Da and linear PEI 25000 Da were purchased from Polysciences, Inc. (Warrington, PN). Linear PEI 400, branched PEI 800 and 25000 Da, and cholesteryl chloroformate were purchased from Aldrich, Inc. (Milwaukee, Wis.); Methyl-PEG-NHS 3400 Da, Methyl-PEG-NHS 1,000 Da, and NH₂—PEG-COOH 3400 Da were purchased from Nectar, Inc. (Huntsville, Ala.). Methyl-PEG-NHS 330, Methyl-PEG-NHS 650, and Amino dPEG₄™ acid were purchased from Quanta Biodesign, Inc. (Powell, Ohio). 2-dioleoyl-sn-glycero-3-phosphoethanolamine (DOPE) was purchased from Avanti Polar Lipids (Alabaster, Ala.). Anhydrous chloroform; ethyl ether, tetrahydrofuran, ethyl acetate, and acetone were purchased from Sigma (St. Louis, Mo.).

### EXAMPLE 1

Synthesis of PPC Consisting of PEG 550, Branched PEI 1800, and Cholesteryl Chloroformate

This example illustrates the preparation of PPC consisting of PEG 550, branched PEI 1800, and Cholesteryl chloroformate.

One gram of branched polyethyleneimine (PEI) 1800 Da (0.56 mM) was dissolved in 5 ml of chloroform and placed in a 100 ml round bottom flask and stirred for 20 minutes at room temperature. Three hundred eighty milligrams of cholesteryl chloroformate (0.84 mM) and 500 mg of poly(ethylene glycol)(PEG) (mw 550 Da)(0.91 mM) were dissolved in 5 ml chloroform and transferred to an addition funnel which was located on the top of the round bottom flask containing the PEI solution. The mixture of cholesteryl chloroformate and PEG in chloroform was slowly added to PEI solution over 5-10 minutes at room temperature. The solution was stirred for an additional 4 hrs at room temperature. After removing the solvent by a rotary evaporator, the remaining sticky material was dissolved in 20 ml ethyl acetate with stirring. The product was precipitated from the solvent by slowly adding 20 ml n-Hexane; the liquid was decanted from the product. The product was washed two times with a 20 ml mixture of ethyl acetate/n-Hexane (1/1; v/v). After decanting the liquid, the material was dried by purging nitrogen gas for 10-15 minutes. The material was dissolved in 10 ml of 0.05N HCl to prepare the salt form of the amine groups. The aqueous solution was filtered through 0.2 pm filter paper. The final product was obtained by lyophilization.

For confirmation, the product was analyzed by the ¹H-NMR (Varian Inc., 500 MHz, Palo, Alto, Calif.). A sample was dissolved in chloroform-d for the NMR measurement. The NMR peaks were analyzed by carrying out characterization of the presence of three components, Cholesterol, PEG, and PEI. The NMR results are as follows: ¹H NMR (500 MHz, chloroform-d1) δ ˜0.65 ppm (3H of CH₃ from cholesterol); δ ˜0.85 ppm (6H of (CH₃)₂ from cholesterol); δ ˜0.95 ppm (3H of CH₃ from cholesterol); δ ˜1.10 ppm (3H of CH₃ from cholesterol); δ ˜0.70˜2.50 ppm (4H from CH₂—CH₂ and CHCH₂ from cholesterol); δ˜5.30 ppm (1H from ═CH— from cholesterol); δ 2.50˜3.60 ppm (176H from N—CH₂—CH₂—N from PEI); and δ ˜3.7 ppm (23H from OCH₂CH₂—O from PEG). The representative peak of each material was calculated by divided the number of hydrogens, and then considered the conjugation ratios. The molar ratio of this example showed that 3.0 moles of PEG and 1.28 moles of cholesterol were conjugated to one mole of PEI molecules.

### EXAMPLE 2

Synthesis of PPC Consisting of PEG 330, Branched PEI 1800, and Cholesteryl Chloroformate

This example illustrates the preparation of PPC consisting of PEG 330, branched PEI 1800, and Cholesteryl chloroformate.

One hundred eighty milligrams of branched PEI 1800 (0.1 mM) was dissolved in 4 ml of chloroformate for 30 minutes at room temperature. Seventy milligrams of cholesteryl chloroformate (0.14 mM) and 48 mg PEG 330 (0.14 mM) were dissolved in 1 ml of chloroformate, and slowly added to the PEI solution over 3-10 minutes using a syringe. The mixture was stirred for 4 hrs at room temperature. After addition of 10 ml of ethyl acetate for precipitation, the solution was incubated overnight at −20° C., and then the liquid was decanted from the flask. The remaining material was washed 2 times with a 5 ml mixture of ethyl acetate/n-Hexane (1/1; v/v). The remaining material was dried by nitrogen purge for 10-15 minutes, dissolved in 10 ml of 0.05N HCl for 20 minutes, and then the solution was filter through a 0.2 μm syringe filter. The aqueous solution was lyophilized by freeze drying to remove water from the polymers.

For confirmation, the product was analyzed by ¹H-NMR (Varian Inc., 500 MHz, Palo, Alto, Calif.). A sample was dissolved in chloroform-d for NMR measurement. The NMR peaks were analyzed by carrying out characterization of the presence of three components, Cholesterol, PEG, and PEI. The NMR results are as follows: ¹H NMR (500 MHz, chloroform-d1) δ ˜0.65 ppm (3H of CH₃ from cholesterol); δ ˜0.85 ppm (6H of (CH₃)₂ from cholesterol); δ ˜0.95 ppm (3H of CH₃ from cholesterol); δ ˜1.10 ppm (3H of CH₃ from cholesterol); δ ˜0.70˜2.50 ppm (4H from CH₂—CH₂ and CHCH₂ from cholesterol); δ˜5.30 ppm (1H from ═CH— from cholesterol); δ2.50˜3.60 ppm (176H from N—CH₂—CH₂—N from PEI); and δ˜3.7 ppm (12H from OCH₂CH₂—O from PEG). The representative peaks of each material were calculated by dividing the number of hydrogens, and then considered the conjugation ratios. The molar ratio of this example showed that 0.85 moles of PEG and 0.9 moles of cholesterol were conjugated to one mole of PEI molecules.

### EXAMPLE 3

Synthesis of PPC Consisting of PEG 1000, Linear PEI 25000, and Cholesteryl Chloroformate

This example illustrates the preparation of PPC consisting of PEG 1000, linear PEI 25000, and Cholesteryl chloroformate.

Five hundred milligrams of 25000 Da linear PEI (0.02 mM) was dissolved in 30 ml at 65° C. for 30 minutes. The three-neck flask was equipped with a condensation and addition funnel. A mixture of 200 mg mPEG-NHS 1000 (0.2 mM) and 40 mg cholesteryl chloroformate (0.08 mM) in 5 ml chloroform was slowly added to the PEI solution over 3-10 minutes. The solution was stirred constantly for an additional 4 hr at 65° C., and then volume was reduced to about 5 ml in a rotary evaporator. The solution was precipitated in 50 ml of ethyl ether to remove free cholesterol, the liquid was decanted from the flask, and the remaining material was washed two times with 20 ml of ethyl ether. After drying with pure nitrogen, the material was dissolved in a mixture of 10 ml of 2.0 N HCl and 2 ml of trifluoroacetic acid. The solution was dialyzed against deionized water using a MWCO 15000 dialysis tube for 48 hrs with changing of fresh water every 12 hrs. The solution was lyophilized to remove water.

The sample was dissolved in deuterium oxide for NMR measurement. The NMR peaks were analyzed by carrying out characterization of the presence of three components, Cholesterol, PEG, and PEI. The NMR results are as follows: ¹H NMR (500 MHz, chloroform-d1) δ˜0.65 ppm (3H of CH₃ from cholesterol); (2340H from N—CH₂—CH₂—N from PEI); and δ˜3.7 ppm (91H from OCH₂CH₂—O from PEG). The representative peaks of each material were calculated by dividing the number of hydrogens, and then considered the conjugation ratios. The molar ratio of this example showed that 12.0 moles of PEG and 5.0 moles of cholesterol were conjugated to one mole of PEI molecules.

### EXAMPLE 4

Synthesis of Water-Insoluble Lipopolymer Consisting of PEI 1800 and Cholesteryl Chloroformate

This example illustrates the preparation of water-insoluble lipopolymers.

One gram of PEI (Mw: 1200 Daltons) was dissolved in a mixture of 15 mL anhydrous methylene chloride and 100 μl triethylamine (TEA). After stirring on ice for 30 minutes, 1.2 g of cholesteryl chloroformate solution was slowly added to the PEI solution and the mixture was stirred overnight on ice. The resulting product was precipitated by adding ethyl ether followed by centrifugation and subsequent washing with additional ethyl ether and acetone. Water-insoluble lipopolymer was dissolved in chloroform to give a final concentration of 0.08 g/mL. Following synthesis and purification, the water-insoluble lipopolymer was characterized using MALDI-TOFF MS and ¹H NMR.

The NMR measurement of water insoluble lipopolymer 1200 showed the following results: ¹H NMR (200 MHz, CDCl₃), δ 0.6 (3H of CH₃ from cholesterol); δ 2.5 (230H of —NHCH₂CH₂— from the backbone of PEI); δ 3.1 (72H of ═N—CH₂CH₂—NH₂ from the side chain of PEI); δ 5.3 (1H of ═C═CH—C— from cholesterol). Another peak appearing at δ 0.8, −δ 1.9 was cholesterol. The amount of cholesterol conjugated to the PEI was determined to be about 40%. MALDI-TOF mass spectrometric analysis of the water-insoluble lipopolymer showed its molecular weight to be approximately 1600. The peak appeared from 800 to 2700 and the majority of peaks were around 1600, which is expected since PEI of 1200 Da and cholesterol of 414 (removal of chloride) were used for synthesis. This suggests that the majority of PEACE 1200 synthesized was a 1/1 molar ratio of cholesterol and PEI, although some were either not conjugated or conjugated at a molar ratio of 2/1 (cholesterol/PEI).

### Example 5

Synthesis of Water Soluble Lipopolymer Consisting of PEI 1800 and Cholesteryl Chloroformate Using Primary Amine Group

This example illustrates the preparation of a water-soluble lipopolymer consisting of PEI 1800 and cholesteryl chloroformate.

Three grams of PEI (Mw: 1800 Daltons) was stirred for 30 minutes on ice in a mixture of 10 ml of anhydrous ethylene chloride and 100 μl of triethyamine. One gram of cholesteryl chloroformate was dissolved in 5 ml of anhydrous ice-cold methylene chloride and then slowly added over 30 minutes to the PEI solution. The mixture was stirred for 12 hours on ice and the resulting product was dried in a rotary evaporator. The powder was dissolved in 50 ml of 0.1 N HCl. The aqueous solution was extracted three times with 100 mL of methylene chloride, and then filtered through a glass microfiber filter. The product was concentrated by solvent evaporation, precipitated with a large excess of acetone, and dried under vacuum. The product was analyzed using MALDI-TOF mass spectrophotometry and ¹H NMR. The product was then stored at −20° C. until used.

The NMR results of water soluble lipopolymer 1800 are as follows: ¹H NMR (500 MHz, D₂O+1,4-Dioxane-d₆), δ 0.8 (2.9H of CH₃ from cholesterol); δ 2.7 (59.6 H of —NHCH₂CH₂— from the backbone of PEI); δ 3.2 (80.8H of ═N—CH₂CH₂—NH₂ from the side chain of PEI); δ 5.4 (0.4H of ═C═CH—C— from cholesterol). Another peak appearing at δ 0.8, −δ 1.9 was cholesterol. The amount of cholesterol conjugated to PEI was determined to be about 47%. MALDI-TOFF mass spectrometric analysis of PEACE showed its molecular weight to be approximately 2200. The peak appeared from 1000 to 3500 and the majority of peaks were around 2200. The expected position is 2400, one chloride 35 is removed from PEI 1800+cholesteryl chloroformate 449. This suggests that the majority of PEACE 1800 synthesized was of a 1/1 molar ratio of cholesterol and PEI, although some were either not conjugated or were conjugated at a molar ratio of 2/1 (cholesterol/PEI).

### EXAMPLE 6

Synthesis of Lipopolymer Consisting of PEI 1800 and Cholesteryl Chloroformate Using Secondary Amine Groups

This example illustrates the preparation of a lipopolymer consisting of PEI 1800 and cholesteryl chloroformate using secondary amine groups for cholesterol conjugation to PEI.

Fifty milligrams PEI 1800 was dissolved in 2 mL of anhydrous methylene chloride on ice. Then, 200 μL of benzyl chloroformate was slowly added to the reaction mixture and the solution was stirred for four hours on ice. Following stirring, 10 mL of methylene chloride was added and the solution was extracted with 15 mL of saturated NH₄Cl. Water was removed from the methylene chloride phase using magnesium sulfate. The solution volume was reduced under vacuum and the product (called CBZ protected PEI) was precipitated with ethyl ether. Fifty milligrams of primary amine CBZ protected PEI was dissolved in methylene chloride, 10 mg of cholesterol chloroformate was added, and the solution was stirred for 12 hours on ice. The product (CBZ protected lipopolymer) was precipitated with ethyl ether, washed with acetone, and then dissolved in DMF containing palladium activated carbon as a catalyst under H₂ as a hydrogen donor. The mixture was stirred for 15 hours at room temperature, filtered with Celite®, and the solution volume was reduced by a rotary evaporator. The final product was obtained from precipitation with ethyl ether.

### EXAMPLE 7

Synthesis of Cholesterol Conjugated to PEI Through PEG Spacer

This example illustrates the synthesis of a PEGylated lipopolymer of the present invention wherein a NH₂-PEG-COOH (mw 3400) was used as a spacer between the cholesterol and PEI.

Five hundred milligrams of NH₂-PEG-COOH 3400 (0.15 mM) was dissolved in 5 ml of anhydrous chloroform at room temperature for 30 minutes. A solution of 676 mg of cholesterol chloroformate (1.5 mM) in 1 ml of anhydrous chloroform was slowly added to the PEG solution and then stirred for an additional 4 hrs at room temperature. The mixture was precipitated in 500 ml of ethyl ether on ice for 1 hr, and then washed three times with ethyl ether to remove the non-conjugated cholesterol. After drying with nitrogen purge, the powder was dissolved in 5 ml of 0.05N HCl for acidifying the carboxyl groups on the PEG. The material was dried by freeze drier. One hundred milligrams of PEI 1800 (0.056 mM), 50 mg of DCC, and 50 mg of NHS were dissolved in 5 ml of chloroform at room temperature, the mixture was stirred for 20 min, and then a solution of 380 mg of chol-PEG-COOH in 1 ml of chloroform was slowly added to the PEI solution. After stirring for six hours at room temperature, the organic solvent was removed with a rotary evaporator. The remaining material was dissolved in 10 ml deionized water and purified by FPLC

### EXAMPLE 8

Synthesis of Glycosylated PPC

This example illustrates the synthesis of a sugar based-targeting moiety conjugated to PPC

Two hundred milligrams of PPC consisting of PEG 550, PEI 1800, and Cholesterol (0.05 mM) was glycosylated using 8 mg of α-D-glucopyranosyl phenylisothiocyanate dissolved in DMF. To synthesize galactosylated, mannosylated and lactosylated PPC, α-D-galactopyranosyl phenylisothiocyanate, α-D-mannopyranosyl phenylisothiocyanate, α-D-lactopyranosyl phenylisothiocyanate were used, respectively. The solution was adjusted to a pH of 9 by addition of 1 M Na₂CO₃ and then incubated for 12 hours at room temperature. The glucosylated PPC was dialyzed against 5 mM NaCl for 2 days with a change of fresh deionized water every 12 hrs. The resulting material was filter through a 0.45 μm filter paper, and then freeze dried.

### EXAMPLE 9

Synthesis of Folate Conjugated to PPC

This example illustrates the preparation of a targeting moiety conjugated lipopolymer consisting of PEI 1800, PEG 550, cholesteryl chloroformate, and folate.

Two hundred milligrams of PPC was conjugated with 10 mg of folic acid dissolved in 5 ml of dimethylsulfoxide (DMSO) containing 50 mg of 1,3-Dicyclohexylcarbodiimide (DCC) and 50 mg of N-hydroxysuccinamide (NHS). After 12 hours of stirring, the product (Folate-PPC) was precipitated in 100 ml of ethyl ether, and then the liquid was decanted carefully after remaining for 1 hr at room temperature. The remaining material was dissolved in 10 ml of 1N HCl. The solution was dialyzed against deionized water for two days with a change of fresh deionized water every 12 hr. The solutions were filtered through 0.45 μm filter paper, and then freeze dried.

### EXAMPLE 10

Synthesis of an RGD Conjugated PPC

This example illustrates the preparation of RGD peptide conjugated lipopolymer consisting of PEI 1800, PEG 550, cholesteryl chloroformate, and RGD peptide as a targeting moiety.

Cyclic NH₂-Cys-Arg-Gly-Asp-Met-Phe-Gly-Cys-CO—NH₂ was used as an RGD peptide with an N-terminus. An RGD peptide was synthesized using solid phase peptide synthetic methods with F-moc chemistry. Cyclization was performed overnight at room temperature using 0.01M K₃[Fe(CN)₆] in 1 mM NH₄OAc at a pH of 8.0 and then purification was done with HPLC. One mole of N-terminal amine groups of the RGD peptide was reacted with 2 moles N-succinimidyl 3 (2-pyridyldithio) propionate (SPDP) in DMSO and precipitated with ethyl ether (RGD-PDP). Two hundred milligrams of PPC were reacted with 7 milligrams of SPDP in DMSO for two hours at room temperature. The resulting materials (PPC-PDP) were treated with 0.1 M (−)1,4-Dithio-L-threitol (DTT) followed by separation in a bio-spin column. RGD-PDP was dissolved in DMF and then added to the PPC-PDP solution. After 12 hours of stirring, the resulting material (RGD-PPC) was purified by FPLC. The resulting solution was dialyzed against deionized water for two days followed by volume reduction using a rotary evaporator. The final product was obtained by freeze drying.

### EXAMPLE 11

Amplification and Purification of Plasmids

This example illustrates the preparation of pDNA to be complexed with the lipopolymer prepared in Examples from 1 to 10.

Plasmid pCMV-Luciferase (pCMV-Luc) was used as a reporter gene and pmIL-12 (a plasmid carrying the murine interleukin-12, or mIL-12 gene) as a therapeutic gene. The p35 and p40 sub-units of mIL-12 were expressed from two independent transcript units, separated by an internal ribosomal entry site (IRES), and inserted into a single plasmid, pCAGG. This vector encodes mIL-12 under the control of the hybrid cytomegalovirus induced enhancer (CMV-IE) and chicken β-actin promoter. All plasmids were amplified in E. coli DH5α strain cells, and then isolated and purified by QIAGEN EndoFree Plasmid Maxi Kits (Chatsworth, Calif.). The plasmid purity and integrity was confirmed by 1% agarose gel electrophoresis, followed by ethidium bromide staining. The pDNA concentration was measured by ultraviolet (UV) absorbance at 260 nm.

### EXAMPLE 12

Preparation of Liposomes

This example illustrates the preparation of lipopolymer/pDNA complexes, wherein the lipopolymers are from the Examples 1-10.

PPC was dissolved in anhydrous methyl alcohol in a round bottom flask and neutral lipid (e.g., cholesterol, DOPE) was added in molar ratios of 1/1, 1/2 and 2/1. The mixture was stirred for around 1 hr at room temperature until becoming clear solution. The clear solution was rotated on a rotary evaporator at 30° C. for 60 minutes until resulting in thin translucent lipid films in the surface of the round bottom flask. The flasks were covered with punctured-parafilm and the lipid film was dried overnight under vacuum. The films were hydrated in 5 mL of sterile water to give a final concentration of 5 mM for the PPC. The hydrated films were vortexed vigorously for 10-20 minutes at room temperature for dispersing in water, and then the dispersed material was more dispersed by ultrasonication in a bath of ultra-sonicator for 30 minutes at room temperature. The dispersed solution was filtered through 450 nm filters and then following passed through 200 nm filters for removing big size particles.

### EXAMPLE 13

Preparation of Water Soluble PPC/pDNA and Water Insoluble PPC:DOPE/pDNA Complexes

This example illustrates the formation of water soluble PPC/pcDNA and PPC:DOPE/pDNA complexes.

The water soluble PPC and PPC:DOPE liposomes and the pDNA prepared in Example 11 were diluted separately with 5% lactose to a volume of 250 μl each, and then the pDNA solution was added to the liposomes under mild vortexing. Complex formation was allowed to proceed for 30 minutes at room temperature. To study the effect of charge ratios for an effective gene transfer, water soluble PPC/pDNA and PPC:DOPE liposomes/pDNA complexes were prepared at N/P ratios ranging from 5/1 to 50/1(N/P). Following complex formation, the osmolality and pH of the PPC:DOPE/pDNA complexes were measured.

The water soluble PPC/pDNA and PPC:DOPE liposomes/pDNA complexes formulated at several N/P ratios were diluted five times in the cuvette for the measurement of the particle size and ζ potential of the complexes. The electrophoretic mobility of the samples was measured at 37° C., pH 7.0 and 677 nm wavelength at a constant angle of 15° with ZetaPALS (Brookhaven Instruments Corp., Holtsville, N.Y.). The zeta potential was calculated from the electrophoretic mobility based on Smoluchowski's formula. Following the determination of electrophoretic mobility, the samples were subjected to mean particle size measurement.

The mean particle size of the water soluble PPC/pDNA complexes was shown to be within the same range of the particle sizes of the composition of PPC which is 90-120 nm. Overall, these complexes had a narrow particle size distribution.

The zeta potential of these complexes was in the range of 20 to 40 mV, and increased with an increase in the N/P ratio (FIG. 5). In addition, the particle size of the PPC/pDNA complexes was shown to be homogenous with a range of 80-120 nm in their diameters. The distribution of particle sizes was not affected greatly by the N/P ratio change (FIG. 5).

### EXAMPLE 14

Gel Retardation Assay for Confirming PPC/pDNA Complexes

This example illustrates confirmation of the complexation between PPC and pDNA by gel retardation assay.

Briefly, various amount of PPC were complexed with pDNA for evaluation of the complexation ability at N/P ratios from 10/1 to 40/1, in the presence of 5% lactose (w/v) to adjust the osmolality to 290˜300 mOsm. The complexes were electrophoresed on a 1% agarose gel. As illustrated in FIG. 4, the positively charged PPC makes strong complexes with the negatively charged phosphate ions on the sugar backbone of DNA. There was not detected any free DNA detected on the screen in the N/P range of 10/1 to 40/1.

### EXAMPLE 15

In Vitro Transfection

This example illustrates the gene transfection to the cultured cells by PPC/pDNA complexes.

PPC/pCMV-Luc complexes were formulated at different N/P ratios in 5% (w/v) lactose for evaluation of their transfection efficiency in 293 T human embryonic kidney transformed cell lines.

In the case of the luciferase gene, 293 T cells were seeded in six well tissue culture plates at 4×10⁵ cells per/well in 10% FBS containing RPMI 1640 media. The cells achieved 80% confluency within 24 hours after which they were transfected with water soluble PPC/pDNA complexes prepared at different PEG ratios containing PPC ranging from 0.2 to 2.5 moles of PEG per PEI molecules. The total amount of DNA loaded was maintained constant at 2.5 μg/well and transfection was carried out in absence of serum. The cells were incubated in the presence of the complexes for five hours in a CO₂ incubator followed by replacement of 2 ml of RPMI 1640 containing 10% FBS and incubation for an additional 36 hours. The cells were lysed using 1× lysis buffer (Promega, Madison, Wis.) after washing with cold PBS. Total protein assays were carried out using a BCA protein assay kit (Pierce Chemical Co, Rockford, Ill.). Luciferase activity was measured in terms of relative light units (RLU) using a 96 well plate Luminometer (Dynex Technologies Inc, Chantilly, Va.). The final values of luciferase were reported in terms of RLU/mg total protein. Both naked DNA and untreated cultures were used as positive and negative controls, respectively. As illustrated in FIG. 6, and 7, the transfection efficiency of PPC was decreased by increasing PEG amounts per molecule of PPC. However, in in vivo the inclusion of PEG increased the transfection activity (Example 16).

### EXAMPLE 16

In Vivo Gene Transfer by Local Administration of PPC/DNA Complexes

This example illustrates gene expression after administration to a local site of tumor by PPC/pDNA complexes.

Depending upon their physico-chemical properties (e.g., particle size and surface charge) the PPC/pDNA complexes can be employed for local and systemic gene delivery. For gene targeting to distal tissues (e.g., lung, liver, spleen and distal tumors) by systemic administration the transfection complexes must be stable in the blood circulation and escape recognition by the immune system.

This example illustrates the application of the present invention, PPC, as the gene carrier for local gene delivery to solid tumors. 4T 1 breast cancer cells (1×10⁶ cells) were implanted on the flanks of in Balb/c mice to create solid tumors. 7-10 days after implantation the tumors were given 30 ul (6 ug) of luciferase plasmid (0.2 mg/ml) complexed with PEI-Chol or PPC at various PEG to PEI molar ratios in the range of 0.6:1-18:1. The plasmid/polymer complexes were prepared at an N/P ratio of 16.75. Twenty four hours after DNA injection the tumors were harvested, homogenized, and the supernatant was analyzed for luciferase activity as a measure of gene transfer. The results from the tumor gene transfer study are shown in FIG. 7. Addition of PEG increased the activity of the PEI-Chol polymer. The maximum gene transfer activity was achieved at PEG:PEI molar ratio of around 2:1. The PPC polymer at various PEG:PEI molar ratios was also tested with a therapeutic gene, IL-12. As shown in FIG. 8, PPC IL-12 gene transfer into 4T1 tumors was achieved at PEG:PEI ratios of 2-3.5.

### EXAMPLE 17

In Vivo Gene Transfer by Systemic Administration of PPC Liposome/DNA Complexes

This example illustrates the application of the PPC liposomes for systemic gene delivery.

The PPC liposomes with cholesterol were prepared as described in Example 12, and complexed with luciferase plasmids for tail vein administration into mice. Twenty four hours after gene injection the lungs were harvested and homogenized in physiological buffer. An aliquot of the lung tissue supernatant was analyzed for luciferase expression. The luciferase activity in the control and PPC liposome/DNA injected animals is shown in FIG. 9. The enhancement of PPC activity by neutral lipid is presumably due to increased destabilization of the endosomal membrane. In a separate experiment, PPC liposomes were complexed with IL-12 plasmids to test their activity for inhibition of lung metastases following intravenous injection. Renal carcinoma cells were injected intravenously into BALB/c mice to generate pulmonary metastases. 300 ul of PPC liposome/pmIL-12 complexes containing 60 ug of mIL-12 plasmid were injected into tail vein on 6th and 13th day after tumor implantation. The animals were sacrificed on day 24 and tumor nodules in lungs were counted. FIG. 10 shows significant inhibition of pulmonary metastases after intravenous administration of IL-12 plasmid/PPC liposome complexes.

Thus, among the various embodiments taught there has been disclosed a composition comprising a novel cationic lipopolymer and method of use thereof for delivering bioactive agents, such as DNA, RNA, oligonucleotides, proteins, peptides, and drugs, by facilitating their transmembrane transport or by enhancing their adhesion to biological surfaces. It will be readily apparent to those skilled in the art that various changes and modifications of an obvious nature may be made without departing from the spirit of the invention, and all such changes and modifications are considered to fall within the scope of the invention as defined by the appended claims.

It is to be understood that the above-referenced arrangements are only illustrative of the application of the principles of the present invention. Numerous modifications and alternative arrangements can be devised without departing from the spirit and scope of the present invention. While the present invention has been shown in the drawings and is fully described above with particularity and detail in connection with what is presently deemed to be the most practical and preferred embodiments(s) of the invention, it will be apparent to those of ordinary skill in the art that numerous modifications can be made without departing from the principles and concepts of the invention as set forth in the claims.

We claim:

1. A biocompatible cationic lipopolymer comprising a polyethylenimine (PEI), a lipid, and a biocompatible hydrophilic polymer spacer, wherein the lipid is attached to the PEI back bone via the biocompatible hydrophilic polymer spacer by a covalent bond.

2. The cationic lipopolymer of claim 1, wherein the polyethylenimine has a linear or branched configuration with a molecular weight of between 100-500,000 Daltons.

3. The cationic lipopolymer of claim 1, wherein the covalent bond is an ester, amide, urethane or di-thiol bond.

4. The cationic lipopolymer of claim 1, wherein the lipid is cholesterol, cholesterol derivatives, C₁₂ to C₁₈ fatty acids, or fatty acid derivatives.

5. The cationic lipopolymer of claim 1, wherein the biocompatible hydrophilic polymer is polyethylene glycol (PEG) having a molecular weight of between 50 to 20,000 Daltons.

6. The cationic lipopolymer of claim 1, wherein molar ratio of PEI to the hydrophilic polymer is within a range 1:0.1 to 1:500.

7. The cationic lipopolymer of claim 1, wherein molar ratio of the PEI to the lipid is within a range of 1:0.1 to 1:500.

8 The cationic lipopolymer of claim 1 further comprises a targeting moiety which is covalently attached to the PEI back bone directly or through a hydrophilic spacer.

9. The cationic lipopolymer of claim 8, wherein the targeting moiety is selected from the group consisting of transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents, lysosomotrophic agents, and nucleus localization signals (NLS).

10. The cationic lipopolymer of claim 8, wherein the covalent bond is an ester, amide, urethane, or dithiol bond.

11. The cationic lipopolymer of claim 8, wherein the molar ratio of the cationic lipopolymer and the targeting moiety is within a range of 1:0.1 to 1:100.

12. A cationic lipopolymer comprising a polyethylenimine (PEI), a lipid, and a biocompatible hydrophilic polymer, wherein the lipid and the biocompatible hydrophilic polymer are directly and independently attached to the PEI backbone by a covalent bond.

13. The cationic lipopolymer of claim 12, wherein the polyethylenimine has a linear or branched configuration with a molecular weight of between 100-500,000 Daltons.

14. The cationic lipopolymer of claim 12, wherein the covalent bond is an ester, amide, urethane, ether, carbonate or di-thiol bond.

15. The cationic lipopolymer of claim 12, wherein the lipid is cholesterol, cholesterol derivatives, C₁₂ to C₁₈ fatty acids, or fatty acid derivatives.

16. The cationic lipopolymer of claim 12, wherein the biocompatible hydrophilic polymer spacer is polyethylene glycol (PEG) having a molecular weight of between 50 to 20,000 Daltons.

17. The cationic lipopolymer of claim 12, wherein the molar ratio of the PEI to the lipid is within a range of 1:0.1 to 1:500.

18. The cationic lipopolymer of claim 12 further comprises a targeting moiety which s covalently attached to the PEI backbone directly or through a hydrophilic spacer.

19. The cationic lipopolymer of claim 18, wherein the targeting moiety is selected from the group consisting of transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, folate, galactose, lactose, and thrombomodulin, fusogenic agents, lysosomotrophic agents, and nucleus localization signals (NLS).

20. The cationic lipopolymer of claim 18, wherein the covalent bond is an ester, amide, urethane, or dithiol bond.

21. The cationic lipopolymer of claim 18, wherein the molar ratio of the cationic lipopolymer and the targeting moiety is within a range of 1:0.1 to 1:100.

22. A complex formed between a nucleic acid and a cationic lipopolymer of claim 1 in a N/P (nitrogen atoms on polymer/phosphate atoms on DNA) ratio within a range of 0.1/1 to 500/1.

23. A complex formed between a nucleic acid and a cationic lipopolymer of claim 8 in a N/P (nitrogen atoms on polymer/phosphate atoms on DNA) ratio within a range of 0.1/1 to 500/1.

24. A complex formed between a nucleic acid and a cationic lipopolymer of claim 12 in a N/P (nitrogen atoms on polymer/phosphate atoms on DNA) ratio within a range of 0.1/1 to 500/1.

25. A complex formed between a nucleic acid and a cationic lipopolymer of claim 18, in a N/P (nitrogen atoms on polymer/phosphate atoms on DNA) ratio within a range of 0.1/1 to 500/1.

26. A liposome comprising a biocompatible cationic lipopolymer of claim of 1 and a helper lipid in a molar ratio within a range of 1:0.1 to 1:500.

27. The liposome of claim 26, wherein the helper lipid is a member selected from the group consisting of cholesterol, dioleoylphosphatidylethanolamine (DOPE), oleoylpalmitoylphosphatidylethanolamin (POPE), diphytanoylphosphatidylethanolamin (diphytanoylPE), disteroyl-, -palmitoyl-, -myristoylphosphatidylethanolamine and 1- to 3-fold N-methylated derivatives.
